(19) United States
(12) Patent Application Publication
(10) Pub. No.: US 2020/0352778 A1
(43) Pub. Date: Nov. 12, 2020

(54) Appendage Cooling System for Concussion Assessment

(71) Applicant: ANSwers Neuroscience Inc., New York, NY (US)

(72) Inventors: Charles Miller, Port Orange, FL (US); Daniel Doorley, Southampton, NY (US)

(21) Appl. No.: 16/832,430

(22) Filed: Mar. 27, 2020

Related U.S. Application Data
(60) Provisional application No. 62/844,014, filed on May 6, 2019.

(51) Int. Cl. A61F 7/00

(52) U.S. Cl. CPC A61F 7/007; A61F 2007/0096; A61F 2007/0093; A61F 2007/0037; A61F 2007/0075; A61F 2007/0056; A61F 2007/0036; A61F 2007/0086

(57) ABSTRACT

Novel tools and techniques for an appendage cooling and concussion assessment are provided. A system includes a cooling device comprising a cold side and a hot side, a cooling interface configured to be coupled to a patient appendage and to cool the patient appendage, a heatsink coupled to the hot side of the cooling device, one or more temperature sensors, and a controller in communication with the cooling device. The controller may be configured to cool, via the cooling interface, the patient appendage based on a temperature of at least one of the patient appendage or the cooling interface.

### CROSS REFERENCE TO RELATED APPLICATIONS

This application claims priority to U.S. Provisional Patent Application Ser. No. 62/844,014, filed May 6, 2019 by Charles Miller et al. (attorney docket no. 0921.05PR), entitled “Appendage Cooling System for Concussion Assessment,” the entire disclosure of which is incorporated herein by reference in its entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to medical diagnostic systems, and more particularly to systems for appendage cooling concussion assessment systems.

### BACKGROUND

Conventional methods of concussion assessment and evaluation rely on a medical practitioner to observe a patient, and to have a patient perform various activities and/or exercises. Furthermore, medical practitioners rely on patients to report symptoms both physical and cognitive, such as when performing memory assessments or feelings of irritability, stronger emotions, malaise, etc.

Alternative systems for concussion assessment evaluate a patient's cold induced vasodilation (CIVD) response during the hunting reaction, in which appendages and extremities (e.g., hands, fingers, etc.) exposed to cold conditions repeat a process of vasoconstriction and vasodilation. Thus, in some concussion assessments, CIVD is evaluated by chilling a patient's appendages. Typically, this includes immersion of a patient's appendages in an ice bath or cold water. Such methods of assessing CIVD is imprecise and may be uncomfortable for a patient undergoing such assessment.

Accordingly, tools and techniques are provided for appendage cooling in a concussion assessment system.

### BRIEF DESCRIPTION OF THE DRAWINGS

A further understanding of the nature and advantages of the embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic block diagram of an appendage cooling system for concussion assessment, in accordance with various embodiments;

FIG. 2A is a schematic diagram of one configuration of an appendage cooling system for concussion assessment, in accordance with various embodiments;

FIG. 2B is a schematic diagram of an alternative configuration of an appendage cooling system for concussion assessment, in accordance with various embodiments;

FIG. 3A is a schematic diagram of a cooling interface, in accordance with various embodiments;

FIG. 3B is a schematic diagram of an alternative cooling interface, in accordance with various embodiments;

FIG. 3C is a schematic diagram of a cooling interface, in accordance with various embodiments;

FIG. 3D is a schematic diagram of a cooling interface, in accordance with various embodiments; and

FIG. 4 is a schematic block diagram of a computer system for the appendage cooling system for concussion assessment, in accordance with various embodiments.

### DETAILED DESCRIPTION OF CERTAIN EMBODIMENTS

The following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

The various embodiments include, without limitation, methods, systems, and/or software products. Merely by way of example, a method might comprise one or more procedures, any or all of which are executed by a computer system. Correspondingly, an embodiment might provide a computer system configured with instructions to perform one or more procedures in accordance with methods provided by various other embodiments. Similarly, a computer program might comprise a set of instructions that are executable by a computer system (and/or a processor therein) to perform such operations. In many cases, such software programs are encoded on physical, tangible, and/or non-transitory computer readable media (such as, to name but a few examples, optical media, magnetic media, and/or the like).

In an aspect, a system is provided for appendage cooling and concussion assessment. The system may include a cooling device, cooling interface, heatsink, one or more temperature sensors, and a controller. The cooling device may have a substantially planar structure with two opposing surfaces, the two opposing surfaces comprising a cold side and a hot side. The cooling interface may be configured to be coupled to a patient appendage and to cool the patient appendage. The heatsink may be coupled to the hot side of the cooling device, and the cooling interface may be coupled to the cold side of the cooling device. The controller may be in communication with the cooling device, and further include a processor; and non-transitory computer readable media comprising instructions executable by the processor to cool, via the cooling interface, the patient appendage based on a temperature of at least one of the patient appendage or the cooling interface.

In another aspect, an apparatus for appendage cooling and concussion assessment is provided. The apparatus may include a processor, and non-transitory computer readable media comprising instructions executable by the processor to perform various actions. The instructions may be executed by the processor to obtain, via one or more temperature sensors, a temperature of a cooling interface, obtain, via the one or more temperature sensors, a temperature of the patient appendage, and control, via a cooling device, the temperature of the cooling interface, based on the temperature of at least one of the patient appendage or the cooling interface. The instructions may further be executed by the processor to cool, via the cooling interface, the patient appendage.

In a further aspect, a method for appendage cooling and concussion assessment is provided. The method includes coupling a cooling device to a cooling interface and cooling the cooling interface with the cooling device. The method further includes placing, via the cooling interface, a patient appendage into contact with the cooling device, and cooling, via the cooling interface, the patient appendage based on a temperature of at least one of the patient appendage or the cooling interface.

Various modifications and additions can be made to the embodiments discussed without departing from the scope of the invention. For example, while the embodiments described above refer to specific features, the scope of this invention also includes embodiments having different combination of features and embodiments that do not include all the above described features.

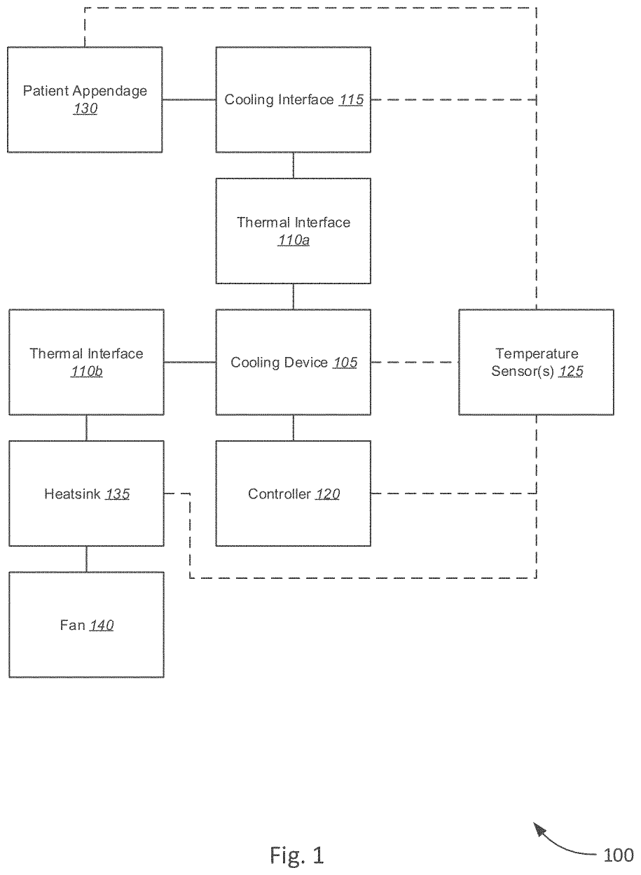

FIG. 1 is a schematic block diagram of an appendage cooling system 100 for concussion assessment, in accordance with various embodiments. The system 100 includes a cooling device 105, a first thermal interface 110a and second thermal interface 110b (collectively “thermal interfaces 110”), cooling interface 115, controller 120, one or more temperature sensors 125, heatsink 135, and fan 140. It should be noted that the various components of the system 100 are schematically illustrated in FIG. 1, and that modifications to the system 100 may be possible in accordance with various embodiments.

In various embodiments, the cooling device 105 may be coupled, via the first thermal interface 110a, to the cooling interface 115. The cooling device 105 may further be coupled, via the second thermal interface 110b, to a heatsink 135. A controller 120 may be coupled to the cooling device 120. The cooling interface 115 may be coupled to a patient appendage 130, and one or more temperature sensors 125 may be coupled to the cooling interface 115 and/or patient appendage 130. In some embodiments, the controller 120 may further be coupled to the one or more temperature sensors 125. In some further embodiments, the one or more temperature sensors may further be coupled to the heatsink 135.

In various embodiments, the cooling device 105 may include various types of coolers and/or cooling devices. For example, the cooling device 105 may include, without limitation, a thermoelectric cooler, evaporative cooling device, a vapor-compression refrigeration system, or other alternative types of cooling devices, as known to those skilled in the art. The cooling device 105 may be configured to be coupled to a cooling interface 115 via which a patient appendage 130 may be cooled. Accordingly, in some embodiments, a thermal interface 110, such as first thermal interface 110a, may be provided to couple the cooling device 105 to the cooling interface 115. In some embodiments, the thermal interface 110 may include, for example, any suitable type of thermal interface material (TIM) as known to those in the art. For example, the thermal interface 110 may include, without limitation, one or more of a thermal grease, thermal paste, thermal adhesive, and/or thermally conductive pads. Thus, the thermal interface 110 may include any suitable TIM configured to increase thermal coupling (e.g., increase the efficiency of heat transfer) between the cooling device 105 and the cooling interface 115, and in some embodiments, between the cooling device 105 and the heatsink 135.

In some embodiments, the cooling device 105 may be a thermoelectric cooling (TEC) device (e.g., a Peltier device), heat pump or other heat transfer device, or any other cooling device. The cooling device 105 may, thus, resemble a planar structure with opposing flat surfaces, a first flat surface being the cool side, and a second flat surface being the hot side. Thus, to cool the patient appendage, the cool side of the cooling device 105 may be coupled to the cooling interface 115, via the first thermal interface 110a. The hot side of the cooling device 105 may be coupled to the heatsink 135, via the second thermal interface 110b.

In alternative arrangements, the thermal interface 110 may further include a circulating coolant material, including liquid and/or gas, to transfer heat between the cooling interface 115 and the cooling device 105, and from the cooling device to the heatsink 135. The thermal interface 110 may further include, without limitation, pumps, hoses, and other devices configured to circulate the coolant between the cooling device 105 and the cooling interface 115 and/or heatsink 135. For example, in some embodiments, the cooling device 105 may be a TEC device, with the cool side thermally coupled to the cooling interface 115 via the first thermal interface 110a, which may be coolant circulated through the cooling interface 115 via a fluid pump. The hot side of the TEC device may similarly be thermally coupled to a heatsink 135 via a second thermal interface 110b, in which coolant may similarly be circulated through and/or to the heatsink 135 via the fluid pump. In further embodiments, different types of thermal interfaces 110a, 110b may be combined, including circulating coolant on one of the cold or hot side, and TIM on the other of the cold or hot side.

In yet further embodiments, the cooling device 105 may include a vapor-compression refrigeration system. Accordingly, in some examples, the cooling device 105 may include one or more of a compressor, pump, evaporator, and/or condenser. Refrigerant, liquid and/or gaseous, may accordingly be pumped, via thermal interfaces 110a, 110b, and brought into contact with cooling interface 115 and/or heatsink 135 respectively. As will be appreciated by those skilled in the art, in some embodiments, the heatsink 135 may, accordingly, be part of the cooling device 105 as an evaporator and/or condenser.

In various embodiments, heat removed from the cooling interface 115, and/or generated by the cooling device 105 may be dissipated via the heatsink 135. Heat stored in the heatsink 135 may, for example, be cooled and/or dissipated into the air via the fan 140. The heatsink 135 may include, without limitation, various types of passive heat exchangers configured to dissipate heat into a fluid medium, such as the air, as known to those in the art. In alternative embodiments, the heatsink 135 may be part of an active heat exchanger, such as a coolant-air and/or water-air radiators, and other suitable solutions coolant circulation configurations of the system 100.

In various embodiments, cooling interface 115 may, accordingly, be configured to be coupled to the patient appendage 130 (such as a hand, palm, fingers, feet, arms, legs, etc.) to facilitate the efficient cooling of the patient appendage via surface contact (e.g., skin contact) with the patient appendage 130. Accordingly, in various embodiments, the cooling interface 115 may be adapted to a specific appendage. In some examples, the cooling interface 115 may include a three-dimensional hemi-ellipsoid structure configured to be gripped and/or make contact with the patient appendage, for example, a hand. Accordingly, the cooling interface 115 may, in some examples, be configured to be in contact with the palm of a hand, and/or both the palm and fingers of a hand. In some embodiments, the cooling interface 115 may have an irregular, hemi-ellipsoidal three-dimensional shape, such as a hemi-egg shape, to fit the patient appendage 130 more ergonomically. In further embodiments, the cooling interface 115 may be a handle with a joystick-like (e.g., aircraft control column) and/or pistol grip-like structure to be gripped in the hand, a mitten, glove, or splint-like structure into which a hand may be inserted, a boot or sock into which a foot may be inserted, etc. Accordingly, the cooling interface 115 may be configured to facilitate contact with the patient appendage 130 such that the patient appendage 130 may be efficiently cooled. In some further embodiments, the cooling interface 115 may be configured to be secured to the patient appendage 130 until cooling of the patient appendage 130 has been completed. For example, in some embodiments, the cooling interface 115 may include, without limitation, a protective shroud, strap, or buckle which may be configured to maintain contact between the appendage 130 and the cooling interface 115. For example, the protective shroud may be a cover surrounding the cooling interface 115 that may be opened to allow access to a patient appendage 130 to be positioned in the cooling interface 115 and closed to help maintain contact between the appendage 130 and cooling interface 115. Thus, in some embodiments, the cooling interface 115 may include further features configured to prevent the patient appendage 130 from inadvertently moving, breaking contact with cooling interface, or being removed from the cooling interface 115.

In various embodiments, the system 100 may further include one or more temperature sensors 125. Temperature sensors 125 may include, without limitation, various types of thermometers, such as thermistors, thermocouples, resistance thermometers, optical thermometers (e.g., infrared thermometers), or other suitable temperature sensors as known to those in the art. In some embodiments, one or more temperature sensors 125 may be coupled to one or more of the cooling interface 115, cooling device 105, controller 120, heatsink 135, and/or patient appendage 130.

For example, in some embodiments, the cooling interface 115 may include one or more temperature sensors 125. Thus, one or more temperature sensors 125 may be coupled to the cooling interface and configured to detect the temperature of the cooling interface 115 at one or more locations of the cooling interface 115. For example, a first temperature sensor of the one or more temperature sensors 125 may be coupled to a palm area of the cooling interface to monitor a temperature of the cooling interface 115 at a location corresponding to where the palm of the patient appendage 130 comes into contact with the cooling interface 115, while a second temperature sensor of the one or more temperature sensors 125 may be coupled to a fingertip area of the cooling interface 115 and configured to monitor a temperature of the cooling interface 115 at a location corresponding to where the fingertips of the patient appendage 130 comes into contact with the cooling interface 115.

In some embodiments, one or more temperature sensors 125 may be coupled to the patient appendage 130. For example, in some embodiments, one or more temperature sensors 125 may be arranged on the surface of the cooling interface 115 to come into direct contact with the patient appendage 130 and/or elsewhere within cooling interface 115 to detect a temperature of the patient appendage 130. In further embodiments, one or more temperature sensors 125 may be attached directly to the skin of a patient appendage. Accordingly, in various embodiments, one or more temperature sensors 125 may be configured to monitor, detect, or otherwise sense the temperature of a patient appendage 130, a surface temperature of the patient appendage 130, and/or specific parts of the patient appendage 130 (e.g., palms, fingertips, soles, or toes).

In some embodiments, one or more temperature sensors 125 may further be coupled to the cooling device 105 and/or heatsink 135. Accordingly, in some embodiments, the one or more temperature sensors 125 may be configured to monitor, detect, or otherwise determine the temperature of the cooling device 105 and/or heatsink 135. For example, in some embodiments, one or more temperature sensors 125 may be coupled to the cold side of the cooling device 105 to determine a temperature of the cold side of the cooling device 105. Similarly, one or more temperature sensors 125 may be coupled to the hot side of the cooling device 105 to determine the temperature of the hot side. In further embodiments, each of the one or more temperature sensors 125 may further be coupled to the controller 120. Temperatures may be reported, or otherwise transmitted to the controller 120.

In yet further embodiments, other types of physiological sensors may be implemented into the system 100, for example at the cooling interface 115, that may measure other physiological signals. Physiological sensors may include, without limitation, heart rate monitors and pulse oximeters. Physiological data may similarly be reported, or otherwise transmitted to the controller 120. Accordingly, in various embodiments, the controller 120 may be configured to control various components of the system 100 based on temperatures reported by the one or more temperature sensors 125. For example, in some embodiments, controller 120 may be configured to control the cooling device 105 based on the temperature-feedback from the cold side and/or hot side. In some further embodiments, the controller 120 may further be configured to control one or more of the functions of the fan 140, thermal interfaces 110, cooling interface 115, etc. Accordingly, the controller 120 may include hardware, software, or both hardware and software. In some embodiments, the controller 120 may be implemented on, without limitation, one or more server computers, dedicated custom hardware, programmable logic controllers, single board computers, field programmable gate arrays (FPGA), application specific integrated circuits (ASIC), or a system on a chip (SoC).

In various embodiments, the controller 120 may be configured to control the cooling device 105 based on a temperature of the patient appendage 130. For example, the cooling device 105 may be controlled to maintain a first temperature on a cold side of the cooling device 105 until a threshold temperature of the patient appendage 130 is reached. Thus, in various embodiments, the controller 120 may be configured to determine, based on the one or more temperature sensors 125, a temperature of the patient appendage 130. Once the temperature of the patient appendage 130 reaches the threshold temperature, the temperature of the cold side of the cooling device 105 may be set to a second temperature.

In some embodiments, the controller 120 may determine a starting temperature of the patient appendage 130. The starting temperature of the patient appendage 130 may be the temperature of the patient appendage 130 before cooling of the patient appendage 130 by the cooling device 105. In some embodiments, the temperature of the patient appendage 130 may be monitored periodically and/or continuously. Alternatively, one or more temperature measurements may be taken of the patient appendage 130. Thus, the temperature of the cooling device 105 may correspondingly be adjusted periodically and/or continuously, based on the temperature of the patient appendage 130. In some embodiments, the second temperature may be higher than the first temperature. Thus, in some embodiments, as the patient appendage 130 approaches the threshold temperature, the temperature of the cooling device 105 may be increased, so as to prevent overcooling of the patient appendage 130. Alternatively, the second temperature may be lower than the first temperature such that the patient appendage 130 more quickly reaches the threshold temperature.

In various embodiments, the controller 120 may further be configured to determine a rate of cooling of the patient appendage 130. For example, the controller 120 The controller 120 may be configured to control the cooling device 105 based on a rate of cooling of the patient appendage 130. For example, the controller 120 may be configured to determine a first temperature of the patient at a first time, and a second temperature of the patient at a second time. Based on the change between the first temperature and the second temperature over time (between the first time and the second time), the controller 120 may determine a first rate of cooling of the patient appendage 130. In some embodiments, if the rate of cooling is determined to be below a first threshold rate of cooling (e.g., cooling too slowly), the controller 120 may be configured to decrease the temperature of the cooling device. If the rate of cooling is determined to be above a second threshold rate of cooling (e.g., cooling too quickly), the controller 120 may be configured to increase the temperature of the cooling device 105.

In some embodiments, the controller 120 may control the temperature of the cooling device 105 based on a pre-determined algorithm. For example, in some embodiments, the controller 120 may be configured to cause the cooling device 105 to follow a cooling algorithm (e.g., a sequence of temperatures). For example, the cooling device 105 may be set to a first temperature. Once the patient appendage 130 reaches a target temperature, the cooling device 105 may be set to a second temperature. In some embodiments, the cooling device 105 may be configured to adjust the temperature of the cooling device 105 through a sequence of temperatures as the patient appendage 130 reaches a series of one or more target temperatures. In further embodiments, the controller 120 adjust temperature based on time. For example, the controller 120 may cause the cooling device 105 to be cooled to a first temperature at a first time, and a second temperature at a second time. Thus, the temperature of the cooling device 105 may be changed over time by the controller 120.

In yet further embodiments, the controller 120 may be configured to record and/or maintain a log of temperatures of the patient appendage 130 and/or cooling device 105 over time. Thus, in some embodiments, the control device 105 may be configured to adjust a temperature of the cooling device 105 based on one or more historic temperatures of the control device 105 and/or the patient appendage 130. In further embodiments, the controller 120 may further be configured to transmit recorded the one or more historic temperatures (e.g., temperature data) of the cooling device 105 and/or patient appendage 120 to another computer device, such as a server, for further analysis.

In one set of embodiments, the cooling device 105 may be a TEC device with a 40 mm×40 mm footprint (e.g., cold side surface and hot side surface). The controller 120 may be configured to maintain a cold side plate temperature of approximately 0 degrees C. The cooling interface 115 may, in turn, be a metallic hemi-ellipsoidal structure. In some embodiments, the cooling interface 115 may include a structure made of copper. In other embodiments, the cooling interface 115 may include, without limitation, a structure formed from aluminum, steel, or an alloy. A TIM may be utilized as a thermal interface 115 between the cooling device 105 and the cooling interface 115, and heatsink 135 respectively. In some embodiments, the patient appendage 130, such as a hand, may have a nominal temperature of 28 degrees C., and a skin thickness of approximately ⅛ inch (0.003175 m). The heat transfer coefficient (k) of skin is approximately 0.37. Accordingly, the heat transfer rate of skin is given by the formula:

$$Q ( skin ) = \frac{0.37 * .0016 {m}^{2} * 28 ° C .}{.003175 m} = approximately 5.2 W \qquad ( I )$$

The above heat transfer rate provides a rough order of magnitude for heat extraction from the palm into the cooling device 105. In other words, the cooling device 105/cooling interface 115 will reach equilibrium with a human body if the cooling device 105/cooling interface 115 is able to dissipate roughly 5 Watts over the 40 mm×40 mm area of the cooling device 105. A typical human at rest has a metabolic heat production rate of roughly 58 W/m², which is roughly equivalent to 0.092 Watts dissipated over the 40 mm×40 mm area of the cooling device 105, and at a maximum exertion rate, heat production of a human peaks at roughly 582 Watts/m² (approximately 0.93 Watts over 40 mm×40 mm). Using these numbers, a cooling system capable of extracting 1 Watt over the 40 mm by 40 mm plate area is able to effectively cool a human hand (in the area directly in contact with this plate) to approximately 0 degrees C. Accordingly, in some embodiments, the 40 mm by 40 mm surface of the cooling device may be configured to extract in excess of 1 Watt over that area, or roughly the amount of heat that an active adult dissipates through hand and feet, and the initial heat generation from the palm of the hand.

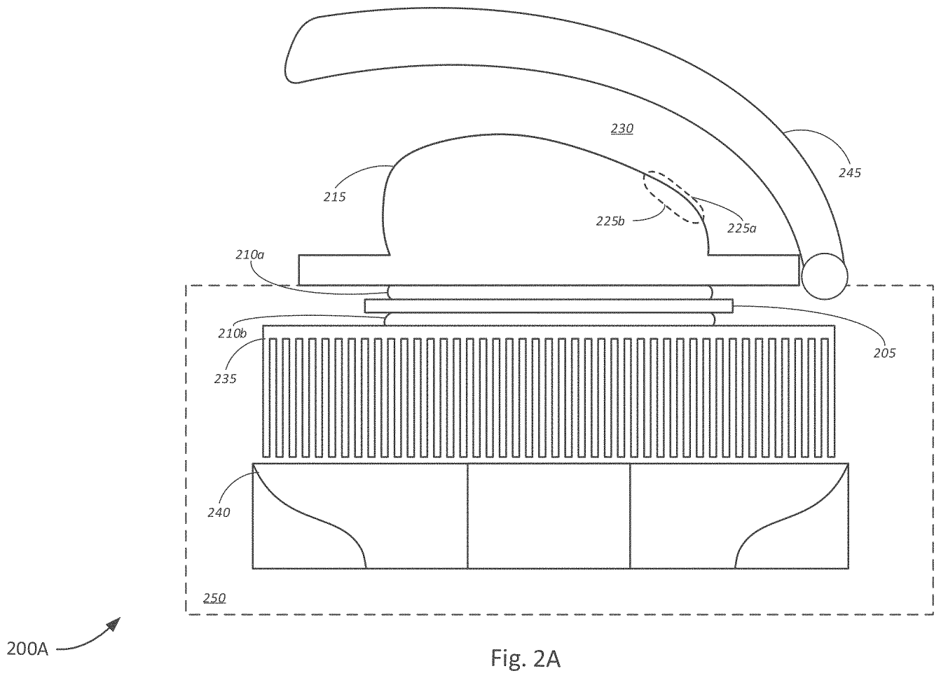

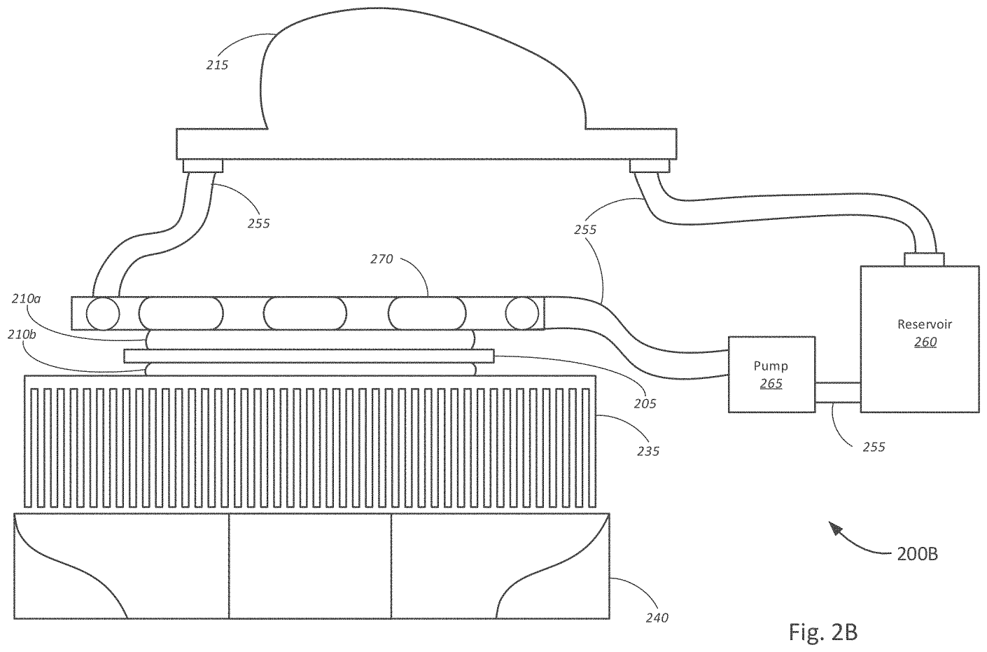

As previously described, the cooling interface 115 may be cooled utilizing different implementations of the thermal interface 110. The various arrangements of the cooling system are depicted in systems 200A-200B, as illustrated by FIGS. 2A-2B. It should be noted, however, that the systems 200A-200B can operate differently in other embodiments (including without limitation those described herein) and in other embodiments, a system different from that depicted by FIG. 2 may be utilized.

FIG. 2A is a schematic diagram of an appendage cooling system 200A for concussion assessment, in accordance with various embodiments. The system 200A includes a sectional, schematic view of cooling device 205, a first thermal interface 210a and second thermal interface 210b (collectively “thermal interfaces” 210), cooling interface 215, first temperature sensor 225a and second temperature sensor 225b (collectively “temperature sensors” 225), appendage compartment 230, heatsink 235, fan 240, protective shroud 245, and enclosure 250. It should be noted that the various components of the system 200A are schematically illustrated in FIG. 2A, and that modifications to the system 200A may be possible in accordance with various embodiments.

In various embodiments of the depicted arrangement, the cooling device 205 may be coupled to the cooling interface 215 via the first thermal interface 210a, and to a heatsink 235 via the second thermal interface 210b. As previously described, in some examples, the cooling device 205 may be a TEC device with a cold side and a hot side. Accordingly, the cold side of the TEC device may be thermally coupled to the cooling interface 215 and the hot side of the TEC device may be thermally coupled to the heatsink 235. Thus, the cooling device 105 may be configured to cool (e.g., remove heat) from the cooling interface 215. Heat generated by the cooling device 105 may then be dissipated via the heatsink 235. Heat in the heatsink 235 may be dissipated into the air via fan 240. In some embodiments, the cooling device 205, heatsink 235, respective thermal interfaces 210, fan 240, and any other components (e.g., one or more temperature sensors 225, controllers, etc.) may be contained within an enclosure 250.

The cooling interface 215 may thus present an exposed surface with which a patient appendage may come into contact with. For example, a patient appendage may be inserted into the appendage compartment 230. In the embodiments depicted, a hand may be inserted into the appendage compartment 230. The cooling interface 215 may, therefore, be configured to be gripped by the hand once the hand is inserted into the appendage compartment. In some embodiments, the appendage compartment 230 may be defined by a volume formed between the cooling interface 215 and protective shroud 245.

In some embodiments, the protective shroud 245 may be a hinged structure, such as a hinged lid, that may open to expose the cooling interface 215 and facilitate placement of the patient appendage. Once the patient appendage has been positioned over the cooling device 215, the protective shroud 245 may be closed to form the appendage compartment 230, and to confine the patient appendage within the appendage compartment 230. Thus, the protective shroud 245 may be configured to prevent inadvertent movement and/or removal of the patient appendage from contact with the cooling interface 215. In some embodiments, the protective shroud 245 may also be cooled by the cooling device 205, for example, via contact with the cooling interface 215.

In some further embodiments, one or more temperature sensors 225 may be operatively coupled to the cooling interface 215 and/or positioned to be operatively coupled with a patient appendage once positioned over the cooling interface 215. For example, a first temperature sensor 225a may be positioned at a fingertip area of the patient appendage (e.g., hand), and configured to monitor a temperature of the fingertip area of the patient appendage. A second temperature sensor 225b may be provided alternatively to or in combination with the first temperature sensor 225a and may be configured to monitor the temperature of the cooling device 215 at a fingertip area of the cooling device 205.

FIG. 2B is a schematic diagram of an alternative arrangement for an appendage cooling system 200B, in accordance with various embodiments. As in FIG. 2A, the system 200B includes cooling device 205, thermal interfaces 210, cooling interface 215, heatsink 235, and fan 240. The system 200B further includes one or more hoses 255, a reservoir 260, pump 265, and cold plate 270. It should be noted that the various components of the system 200B are schematically illustrated in FIG. 2B, and that modifications to the system 200B may be possible in accordance with various embodiments.

In various embodiments, the system 200B may be a configuration that recirculates coolant. Suitable coolants may include liquid or gaseous coolants that may be pumped by pump 265 and recirculated by the system 200B. In some examples, the coolant may be water, oil, or other suitable fluid coolants as known to those in the art. Accordingly, the cooling device 205 may be coupled on the cold side to a cold plate 270 via the first thermal interface 210a, and on the hot side to the heatsink 235 via the second thermal interface 210b. Thus, the cooling device 205 may be configured to cool (e.g., remove heat) from the cold plate 270.

Coolant may be pumped through the cold plate 270. As coolant passes through the cold plate 270, the coolant may be cooled by the cooling device. Accordingly, the cold plate may include one or more of channels, tubes, and/or hoses for directing the coolant. The cooled coolant may then be carried, by one or more hoses 255, to the cooling interface 215. The coolant may be pumped through the cooling interface 215 to cool (e.g., transfer heat) from the cooling interface 215, and the now warmed coolant may flow to the reservoir 260. Thus, in some embodiments, the cooling interface 215 may also include, without limitation, one or more channels, tubes, and/or hoses, both internal and/or external, through which coolant may flow. Warmed coolant from the reservoir 260 may be pumped out, by pump 265, back to the cold plate 270, to once again be cooled by the cooling device 205. Heat generated by the cooling device 205 may then be dissipated via the heatsink 235. Heat in the heatsink 235 may be dissipated into the air via fan 240. In some embodiments, the cooling device 205, heatsink 235, fan 240, respective thermal interfaces 210, one or more hoses 255, reservoir 260, pump 265, cold plate 270, and any other components (e.g., one or more temperature sensors 225, controllers, etc.) may be contained within an enclosure (not shown), such as enclosure 250 of FIG. 2A.

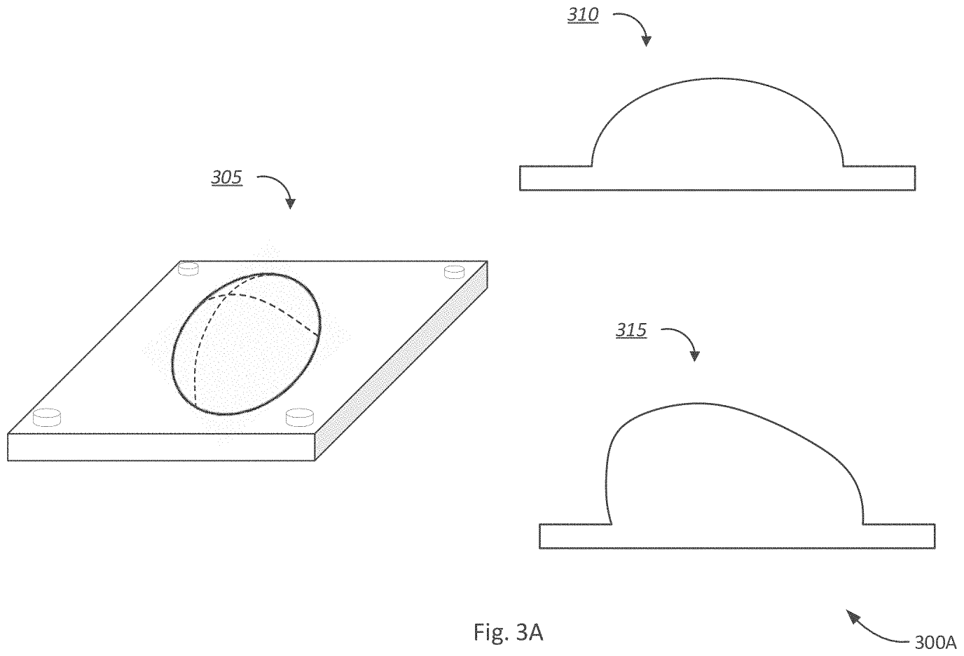

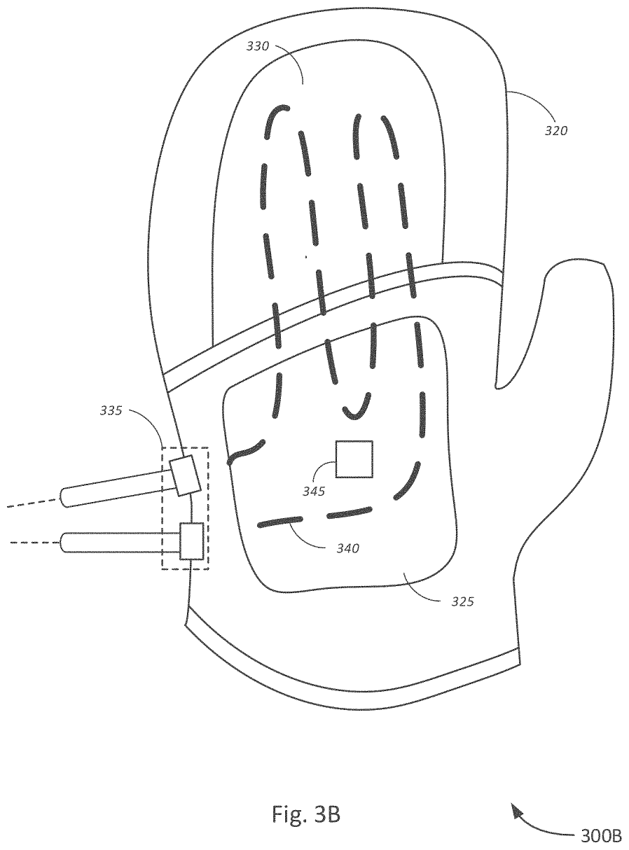

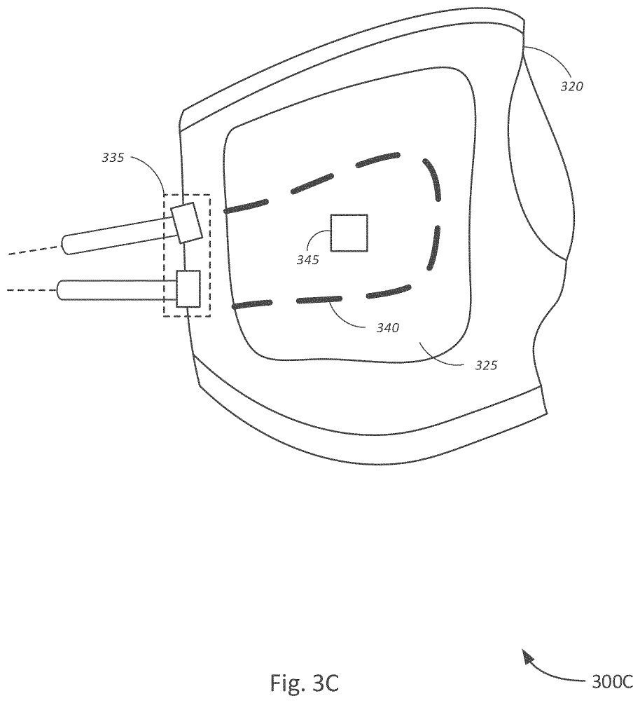

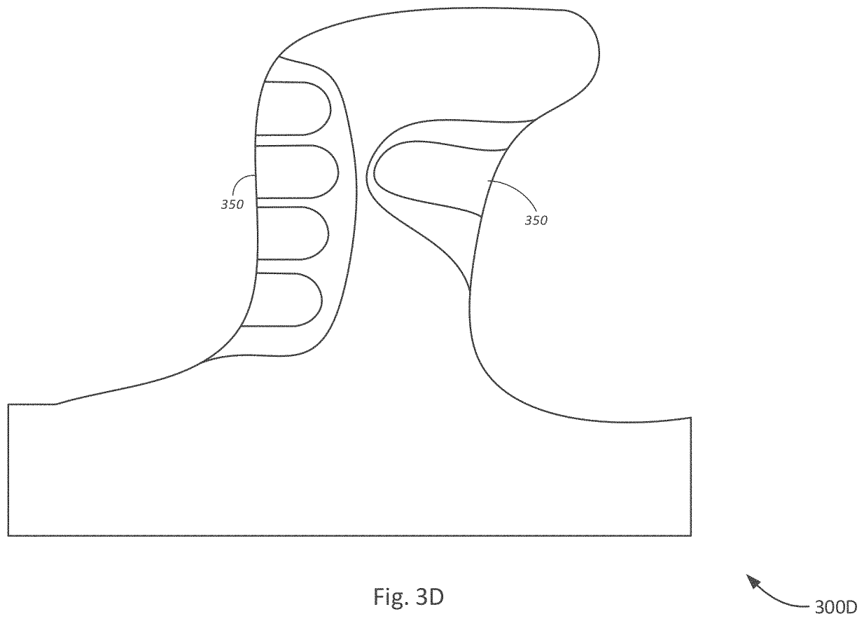

FIGS. 3A-3D illustrate various examples of a cooling interface, as previously described with respect to FIGS. 1-2B. FIG. 3A is a schematic diagram of a hemi-ellipsoidal cooling interface 300A, in accordance with various embodiments. A perspective view 305 illustrates the general hemi-ellipsoidal three-dimensional shape of the cooling interface 300A to be gripped, for example, by a hand. Elevation view 310 illustrates a lengthwise profile side view of the cooling interface 300A according to a first configuration. Elevation view 315 illustrates a lengthwise profile side view of the cooling interface 300A according to a second configuration in which the cooling interface 300A has a hemi-egg shape. In some embodiments, the cooling interface 300A may be a solid structure formed of a material with high thermal conductivity. In some examples, suitable materials may include copper, aluminum, or a suitable alloy. In some embodiments, the cooling interface 300A may include one or more coolant lines (not shown) traversing internally and/or externally around the cooling interface 300A.

FIG. 3B is a schematic diagram of an alternative cooling interface 300B, in accordance with various embodiments. In the embodiments depicted, the body 320 of the cooling interface 300B may be a mitten and/or glove-like structure into which a hand may be inserted.

In various embodiments, the cooling interface 300B may include the body 320, palm area 325, finger area 330, coolant hose connectors 335, one or more coolant lines 340, and one or more temperature sensors 345. In various embodiments, the one or more coolant lines 340 (e.g., tubes/channels) may be integrated into the cooling interface 300B and travel across the palm area 325 and finger area 330, and/or back of the hand (not shown). In some embodiments, the one or more coolant lines 340 may make multiple passes over anticipated areas of contact with the hand. The cooling interface 300B may be filled with a non-freezing medium, such as, without limitation, a clay or gel. The one or more coolant lines 340 may travel through the non-freezing medium, circulating coolant, and cooling the surrounding non-freezing medium. In various embodiments, one or more temperature sensors 345 may be placed in the cooling interface 300B, to monitor temperatures at different areas of the cooling interface 300B, such as the palm area 325. In other embodiments, and as previously described, other temperature sensors (not shown) may be placed in different areas to monitor temperatures of the cooling interface 300B and/or to determine temperatures of a hand placed within the body 320.

FIG. 3C is a schematic diagram of a cooling interface, in accordance with various embodiments. In the embodiments depicted, the body 320 of the cooling interface 300B may be a splint and/or glove-like structure into which a hand may be inserted. As described with respect to the cooling interface 300B, cooling interface 300C may include a body 320, palm area 325, coolant hose connectors 335, one or more coolant lines 340, and one or more temperature sensors 345.

In various embodiments, the one or more coolant lines 340 (e.g., tubes/channels) may be integrated into the cooling interface 300C and travel across the palm area 325 and/or back of the hand (not shown). In some embodiments, the one or more coolant lines 340 may make multiple passes over anticipated areas of contact with the hand, in this example the palm area 325. The cooling interface 300C may similarly be filled with a non-freezing medium as described above. In various embodiments, one or more temperature sensors 345 may be placed in the palm area 325, an area in contact with the back of the hand (not shown), and/or positioned to determine the temperature of a hand placed within the body 320.

FIG. 3D is a schematic diagram of a cooling interface 300D, in accordance with various embodiments. An elevation view of the cooling interface 300D is provided. The body of the cooling interface 300D has a handle shape that resembles a joystick and/or pistol grip. The body of the cooling interface 300D may include one or more grip areas 350 for the thumb and fingers. The cooling interface 300D may, therefore, be gripped by a hand to make contact with the palm of the hand and fingers. Like the cooling interface 300A, in some embodiments, cooling interface 300D may be a solid structure formed of a material with high thermal conductivity. In some examples, suitable materials may include copper, aluminum, or a suitable alloy. In some further embodiments, the cooling interface 300A may include one or more coolant lines (not shown) traversing internally and/or externally around the cooling interface 300D.

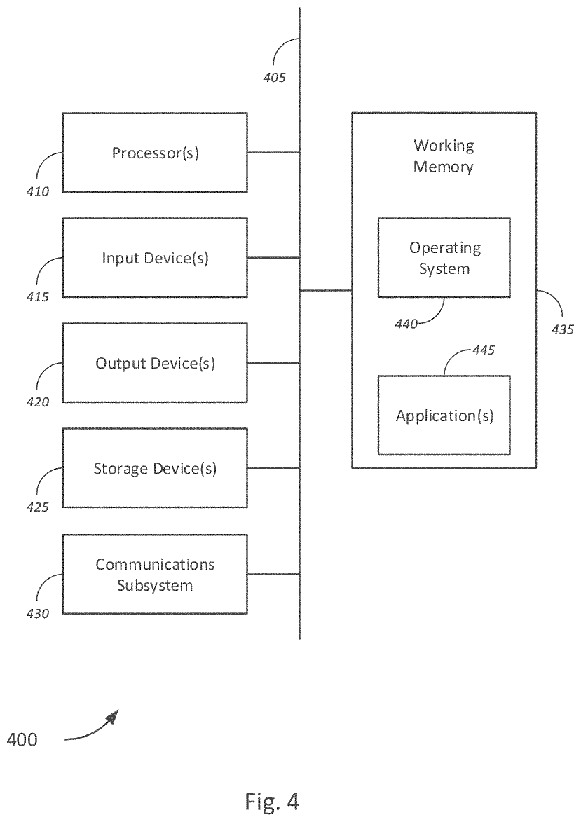

FIG. 4 is a schematic block diagram of a computer system 400 for the appendage cooling system, in accordance with various embodiments. FIG. 4 provides a schematic illustration of one embodiment of a computer system 400, such as a controller for the coolant system, including, cooling device, fans and pumps, and any external computer devices and/or servers that may analyze temperature and/or other physiological data, which may perform the methods provided by various other embodiments, as described herein. It should be noted that FIG. 4 only provides a generalized illustration of various components, of which one or more of each may be utilized as appropriate. FIG. 4, therefore, broadly illustrates how individual system elements may be implemented in a relatively separated or relatively more integrated manner.

The computer system 400 includes multiple hardware elements that may be electrically coupled via a bus 405 (or may otherwise be in communication, as appropriate). The hardware elements may include one or more processors 410, including, without limitation, one or more general-purpose processors and/or one or more special-purpose processors (such as microprocessors, digital signal processing chips, graphics acceleration processors, and microcontrollers); one or more input devices 415, which include, without limitation, a mouse, a keyboard, one or more sensors, and/or the like; and one or more output devices 420, which can include, without limitation, a display device, and/or the like.

The computer system 400 may further include (and/or be in communication with) one or more storage devices 425, which can comprise, without limitation, local and/or network accessible storage, and/or can include, without limitation, a disk drive, a drive array, an optical storage device, solid-state storage device such as a random-access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer system 400 might also include a communications subsystem 430, which may include, without limitation, a modem, a network card (wireless or wired), an IR communication device, a wireless communication device and/or chip set (such as a Bluetooth™ device, an 802.11 device, a WiFi device, a WiMax device, a WWAN device, a Z-Wave device, a ZigBee device, cellular communication facilities, etc.), and/or a LP wireless device. The communications subsystem 430 may permit data to be exchanged with a network (such as the network described below, to name one example), with other computer or hardware systems, between data centers or different cloud platforms, and/or with any other devices described herein. In many embodiments, the computer system 400 further comprises a working memory 435, which can include a RAM or ROM device, as described above.

The computer system 400 also may comprise software elements, shown as being currently located within the working memory 435, including an operating system 440, device drivers, executable libraries, and/or other code, such as one or more application programs 445, which may comprise computer programs provided by various embodiments (including, without limitation, various applications running on the various servers and/or controllers as described above), and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be encoded and/or stored on a non-transitory computer readable storage medium, such as the storage device(s) 425 described above. In some cases, the storage medium might be incorporated within a computer system, such as the system 400. In other embodiments, the storage medium might be separate from a computer system (i.e., a removable medium, such as a compact disc, etc.), and/or provided in an installation package, such that the storage medium can be used to program, configure, and/or adapt a general purpose computer with the instructions/code stored thereon. These instructions might take the form of executable code, which is executable by the computer system 400 and/or might take the form of source and/or installable code, which, upon compilation and/or installation on the computer system 400 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, single board computers, FPGAs, ASICs, and SoCs) might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer or hardware system (such as the computer system 400) to perform methods in accordance with various embodiments of the invention. According to a set of embodiments, some or all of the procedures of such methods are performed by the computer system 400 in response to processor 410 executing one or more sequences of one or more instructions (which might be incorporated into the operating system 440 and/or other code, such as an application program 445) contained in the working memory 435. Such instructions may be read into the working memory 435 from another computer readable medium, such as one or more of the storage device(s) 425. Merely by way of example, execution of the sequences of instructions contained in the working memory 435 might cause the processor(s) 410 to perform one or more procedures of the methods described herein.

The terms “machine readable medium” and “computer readable medium,” as used herein, refer to any medium that participates in providing data that causes a machine to operate in a specific fashion. In an embodiment implemented using the computer system 400, various computer readable media might be involved in providing instructions/code to processor(s) 410 for execution and/or might be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 425. Volatile media includes, without limitation, dynamic memory, such as the working memory 435. In some alternative embodiments, a computer readable medium may take the form of transmission media, which includes, without limitation, coaxial cables, copper wire and fiber optics, including the wires that comprise the bus 405, as well as the various components of the communication subsystem 430 (and/or the media by which the communications subsystem 430 provides communication with other devices). In an alternative set of embodiments, transmission media can also take the form of waves (including, without limitation, radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 410 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 400. These signals, which might be in the form of electromagnetic signals, acoustic signals, optical signals, and/or the like, are all examples of carrier waves on which instructions can be encoded, in accordance with various embodiments of the invention.

The communications subsystem 430 (and/or components thereof) generally receives the signals, and the bus 405 then might carry the signals (and/or the data, instructions, etc. carried by the signals) to the working memory 435, from which the processor(s) 410 retrieves and executes the instructions. The instructions received by the working memory 435 may optionally be stored on a storage device 425 either before or after execution by the processor(s) 410.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to certain structural and/or functional components for ease of description, methods provided by various embodiments are not limited to any single structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described sequentially for ease of description, and unless the context dictates otherwise, various procedures may be reordered, added, and/or omitted in accordance with various embodiments. Moreover, the procedures described with respect to one method or process may be incorporated within other described methods or processes; likewise, system components described according to a specific structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to one embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A system comprising:
a cooling device having a substantially planar structure with two opposing surfaces, the two opposing surfaces comprising a cold side and a hot side;
a cooling interface configured to be coupled to a patient appendage and to cool the patient appendage;
a heatsink coupled to the hot side of the cooling device;
one or more temperature sensors;
a controller in communication with the cooling device, the controller further comprising:
a processor; and
non-transitory computer readable media comprising instructions executable by the processor to:
obtain, via one or more temperature sensors, a temperature of a cooling interface;
obtain, via the one or more temperature sensors, a temperature of the patient appendage;
control, via a cooling device, the temperature of the cooling interface, based on the temperature of at least one of the patient appendage or the cooling interface; and
cool, via the cooling interface, the patient appendage.

2. The system of claim 1, wherein the cooling device is a thermoelectric cooler.

3. The system of claim 1, wherein the cooling interface is coupled to the cold side of the cooling device via a thermal interface material.

4. The system of claim 1, further comprising:
a coolant reservoir configured to hold a coolant;
a pump configured to pump the coolant;
a cold plate coupled to the pump, the cold plate including one or more primary coolant channels configured to allow coolant to be pumped through the one or more coolant channels;
wherein the cooling interface includes one or more secondary coolant channels configured to allow coolant to be pumped through the one or more secondary coolant channels;
wherein the cold plate is coupled to the cooling device, and wherein the cold plate is configured to allow coolant in the one or more primary coolant channels to be cooled by the cooling device.

5. The system of claim 1, wherein the instructions are further executable by the processor to:
maintain, via the cooling device, a first temperature of a cold side of the cooling device;
determine, via the one or more temperature sensors, whether the patient appendage has reached a threshold temperature; and
adjust, via the cooling device, to a second temperature the cold side of the cooling device in response to a determination that the patient appendage has reached the threshold temperature.

6. The system of claim 1, wherein the instructions are further executable by the processor to:
record one or more historic temperatures of the patient appendage over time; and
adjust, via the cooling device, a temperature of a cold side of the cooling device from a first temperature to a second temperature based, at least in part, on the one or more historic temperatures of the patient appendage.

7. The system of claim 1, wherein the cooling interface comprises a hemi-ellipsoidal grip configured to be gripped by the patient appendage.

8. The system of claim 1, wherein the patient appendage is a hand, wherein the cooling interface comprises a fingertip area configured to receive a fingertip of the hand, wherein at least one of the one or more temperature sensors is disposed in the fingertip area of the cooling interface.

9. The system of claim 1, wherein the cooling device further comprises a protective shroud configured to have an open position and a closed position, wherein in the open position, the cooling interface is accessible to gripping by the patient appendage, wherein in the closed position, the protective shroud is configured to mitigate movement of the patient appendage away from contact with the cooling interface.

10. An apparatus comprising:
a processor; and
non-transitory computer readable media comprising instructions executable by the processor to:
obtain, via one or more temperature sensors, a temperature of a cooling interface;
obtain, via the one or more temperature sensors, a temperature of a patient appendage in contact with the cooling interface;
adjust, via a cooling device, the temperature of the cooling interface, based on the temperature of at least one of the patient appendage or the cooling interface; and
cool, via the cooling interface, the patient appendage.

11. The apparatus of claim 10, wherein the cooling interface is coupled to the cooling device, wherein the instructions are further executable by the processor to:
maintain, via the cooling device, a first temperature of a cold side of the cooling device;
determine, via the one or more temperature sensors, whether the patient appendage has reached a threshold temperature; and
adjust, via the cooling device, to a second temperature the cold side of the cooling device in response to a determination that the patient appendage has reached the threshold temperature.

12. The apparatus of claim 10, wherein the instructions are further executable by the processor to:
maintain, via the cooling device, a first temperature of a cold side of the cooling device;
determine, via the one or more temperature sensors, a rate of cooling of the patient appendage; and
adjust, via the cooling device, to a second temperature the cold side of the cooling device based on the rate of cooling of the patient appendage.

13. The apparatus of claim 10, wherein the instructions are further executable by the processor to:
adjust, via the cooling device, a temperature of a cold side of the cooling device from a first temperature at a first time to at least a second temperature at a second time, wherein the first time and the second time are determined according to a control algorithm.

14. The apparatus of claim 10, wherein the instructions are further executable by the processor to:
record one or more historic temperatures of the patient appendage over time.

15. The apparatus of claim 14, wherein the instructions are further executable by the processor to:
adjust, via the cooling device, a temperature of a cold side of the cooling device from a first temperature to a second temperature based, at least in part, on the one or more historic temperatures of the patient appendage.

16. The apparatus of claim 14, wherein the instructions are further executable by the processor to:
transmit the one or more historic temperatures to a server.

17. The apparatus of claim 10, wherein the instructions are further executable by the processor to:
determine, via the one or more temperature sensors, a starting temperature of the patient appendage, wherein the starting temperature of the patient appendage is the temperature of the patient appendage when the cooling interface is at an ambient temperature; and
cooling the cooling device to a first temperature based on the first temperature of the patient appendage.

18. A method comprising:
coupling a cooling device to a cooling interface;
cooling the cooling interface with the cooling device;
placing, via the cooling interface, a patient appendage into contact with the cooling device; and
cooling, via the cooling interface, the patient appendage based on a temperature of at least one of the patient appendage or the cooling interface.

19. The method of claim 18 further comprising:
maintaining, via the cooling device, a first temperature of a cold side of the cooling device;
determining, via the one or more temperature sensors, whether the patient appendage has reached a threshold temperature; and
adjusting, via the cooling device, to a second temperature the cold side of the cooling device in response to a determination that the patient appendage has reached the threshold temperature.

20. The method of claim 18 further comprising:
recording one or more historic temperatures of the patient appendage over time; and
adjusting, via the cooling device, a temperature of a cold side of the cooling device from a first temperature to a second temperature based, at least in part, on the one or more historic temperatures of the patient appendage.
